(19) United States
(12) Patent Application Publication
(10) Pub. No.: US 2004/0215105 A1
(43) Pub. Date: Oct. 28, 2004

(54) Urine deflector shield

(76) Inventor: Vera Jane Maples, Fresno, CA (US)

Correspondence Address: Richard A. Ryan, RYAN & ENGNATH, Suite 104, 8469 N. Millbrook, Fresno, CA, 93720, US

(21) Appl. No.: 10/424,291

(22) Filed: Apr. 28, 2003

(51) Int. Cl. A61B 5/00

(52) U.S. Cl. 600/573

(57) ABSTRACT

A urine deflector shield suitable for removably attaching to a male infant, child or adult to cover part of the male genitalia for deflecting a stream of urine downward to prevent or reduce the likelihood of spraying urine on the person or nearby healthcare workers, equipment and facilities and to facilitate the collection of urine for analysis. The deflector shield has a cover that can be made out of transparent plastic material to facilitate phototherapy. The cover has sides that form an outwardly projecting portion into a tent-like configuration sufficient for deflecting the flow of urine downward and prevent spraying of urine. Extending from the sides are wing members having securing mechanisms, such as adhesive, tape or a belt member, thereon for removably securing the deflector shield to the patient's body generally above the genitalia.

### BACKGROUND OF THE INVENTION

A. Field of the Invention

The present invention relates generally to urine deflectors useful for deflecting urine flows and reducing the likelihood of a person being sprayed by urine. In particular, this invention relates to such urine deflectors that can be placed over the genitalia of male infants, children or adults to deflect urine downward and greatly reduce the likelihood that emergency workers and others will be sprayed by urine during transport, examination, treatment or other medically related activities.

B. BACKGROUND

It is well known by persons in the medical field that the transport, examination or other medical activities pertaining to male patients, particularly infant males, can result in the spraying of urine on the patient and nearby healthcare workers, equipment and facilities. The spraying of urine, and resultant loss of the ability to measure bodily fluids, is often the result of having to transport, examine or medically assist the male patient while he is undressed or partially undressed. Infant males are unable to control their urinary releases and older males, whether children or adult, may have suffered some trauma or have other medical conditions that prevent them from being able to control their urinary functions. Due to the male physiology, the lack or loss of urinary control can result in the urine spraying into the air. At the very least, this urine can land on and contaminate the patient. More often, the urine also sprays on the emergency medical personnel or other healthcare givers that are helping transport or assist the patient. Naturally, being sprayed by urine is an unhealthy and unpleasant experience that is sought to be avoided by healthcare workers. The risks of disease transmission is increased by the potential to receive urine in the mouth or eyes of the healthcare worker. Due to the often emergency nature of the transport or medical assistance and the unknown timing of the urinary release, however, it is often difficult to fully prevent the spraying of urine. Even when there is time or preparation for avoiding such spraying, such as placing something over the genitalia of the male patient, the nature of medical assistance makes it difficult to place a device or covering of any substantial size over the patient. For instance, the placement of an absorbent diaper or other cloth over the patient's genitalia is likely to get in the way or be displaced during the medical treatment, creating the potential for urine spraying.

Another situation where it is common to have problems with males spraying urine is in the nursery area of a hospital. Many times while an infant is in the nursery, particularly newborn infants, a diaper is not used on or over the infant. For instance, it is not at all uncommon for newborn infants to be born with varying degrees of a condition referred to as hyperbilirubinemia (also referred to as jaundice), which is typically treated with phototherapy by placing the uncovered infant under lights (referred to as hyperbili lights). The wavelength of the hyperbili lights react with the infant's body to reduce the level of bilirubin concentration and get rid of the jaundice. The phototherapy acts on the unconjugated bilirubin to a depth of approximately 2 mm from the epidermis to change the bilirubin through photoisomerization into water-soluble lumirubin that is excreted in the urine. Because the effectiveness of the phototherapy is related to the area of the skin exposed to the lights, as well as other factors, it is necessary to directly expose as much of the infant's bare skin as possible to the lights. As such, uncovered male infants can and do spray the nurses and hospital equipment with urine.

In addition, to the problems of contamination, unhealthy and unpleasantness described above, the spraying of urine results in the loss of fluid measurement ability. It is well known that healthcare workers monitor the fluids given to an infant and the bodily fluids, including urine, discharged by the infant to determine the proper amount of food and dosage of medication to give to the infant under their care. Currently, the most common procedure to accomplish this for infants, male and female, is to place a diaper under the infant and attempt to collect the urine in the absorbent portion of the diaper. In this manner, the diaper can be weighed to determine the amount of urine discharged by the infant. To prevent loss of urine due to spraying, some healthcare workers place a small plastic bag or other cover over the male genitalia so that the urine is deflected downward onto the diaper. Besides improving fluid retention and measurement ability, the deflection of the urine downward keeps it off the infant, healthcare workers and hospital equipment.

The deflector cover used by some in the healthcare industry can be as simple as a piece of plastic or a plastic bag. However, often these items are either not readily available or they are not sterile enough to be used on or over an infant's genitalia. In addition, the spray of the urine flow can be sufficiently strong to cause displacement of the cover off the infant. Some medical workers utilize a second diaper over the infant to act as the deflector shield. However, this can be somewhat costly and prevents a problem with treating jaundice infants with the hyperbili lights. To expose the skin to the lights, other medical workers utilize the clear plastic coverings on the tops of bottle nipples to act as a urine deflector to keep from being sprayed and to deflect the urine downward into the diaper. Although the plastic covering of bottle nipples used in hospitals may be sufficiently sterile (i.e., on the underside where it contacts the nipple) to prevent contamination of the infant when it is first removed from the nipple, it results in one of two problems. Either the nipple cover must be set aside for when it is needed on an infant to deflect urine (which requires storing the cover and possible contamination of the cover while stored) or the removal of the nipple cover from the bottle nipple results in a bottle nipple being left unprotected until it is used for feeding (to avoid this problem the nipple is usually discarded once the cover is removed). Either of these problems results in a relatively expensive urine deflector shield and waste associated with the discarded nipples.

The known existing art, both individually and in all appropriate combinations, indicate that the broad concept of utilizing a cover to deflect urine downward to collect the urine and prevent spraying of urine is known. On the other hand, none of the known references teach an easy to use, sterile and sufficiently stable urine deflector shield to deflect urine downward and prevent or at least greatly reduce the likelihood of spraying urine. What is needed is a relatively inexpensive, sterile and sufficient urine deflector shield that is suitable for use to deflect urine downward, prevent or reduce the likelihood of urine being sprayed and facilitate the collection of urine for health monitoring purposes, while allowing as much skin as possible to be exposed for phototherapy treatment of jaundice.

### SUMMARY OF THE INVENTION

The urine deflector shield of the present invention provides the benefits and solves the problems identified above. That is to say, the present invention discloses a urine deflector shield that deflects the flow of urine from a male patient, particularly infants, downward and prevents or greatly reduces the likelihood of urine being sprayed on healthcare workers and other persons. In addition, the present invention improves the ability of healthcare workers and others to collect and monitor the amount of urine discharged by a patient. The present invention provides a urine deflector having a an upright portion and an attachment mechanism suitable for removably attaching the deflector to the patient's body.

In its broadest form, one embodiment of the present invention comprises a shaped cover with one or more sides that form an outwardly projecting portion into a tent-like configuration when the sides are spaced apart. The tent-like configuration is suitable for sufficiently enclosing the male genitalia to direct urine downward where it can be collected, measured and disposed of while preventing or at least greatly reducing the likelihood that urine will be sprayed on the patient and healthcare workers. Each of the sides has a bottom edge thereon with one or more wing members extending from at least one of the sides. In the preferred embodiment, the urine deflector shield cover is made out of a transparent plastic material that is foldable for ease of transport and storing. Alternatively, the cover can be rigid to facilitate formation of the tent-like configuration. In an alternative embodiment, the deflector shield includes a base portion at the bottom edge of the cover. The base portion can be flexible or rigid and it can either be the same as the cover or in contrast to the cover.

A removable securing mechanism is located on at least one of the wing members for removably securing the cover to various parts of the human body to deflect the stream of urine. The securing mechanism can be adhesives (having removable adhesive tab members), tape, double-sided tape or a belt member that goes around the patient's waste. The securing mechanism is designed to hold the deflector shield in position on the patient's body above the genitalia in a manner that prevents it from being easily knocked out of place or being displaced by the flow of urine from the patient. The adhesives or tape should be of the type that is easily and painlessly (as much as possible) removed and does not cause any undue irritation to the patient.

Accordingly, the primary objective of the present invention is to provide a urine deflector shield that is suitable for directing the flow of urine from a male patient downward to prevent or reduce the likelihood that urine will be sprayed a from the male patient and to facilitate the collection of urine for healthcare purposes.

It is also an important objective of the present invention to provide a urine deflector shield that can remain over a male patient's genitalia and not be pushed or knocked of by the flow of urine from the male patient.

It is also an important objective of the present invention to provide a urine deflector shield that has a generally upright portion and one or more attachment mechanism suitable for removably attaching the urine deflector shield to a patient's body.

It is also an important objective of the present invention to provide a urine deflector shield that is relatively inexpensive to make and easy to use.

It is also an important objective of the present invention to provide a urine deflector shield that is suitable for being provided in a substantially sterile condition.

The above and other objectives of the present invention will be explained in greater detail by reference to the figures and the description of the preferred embodiment which follows. As set forth herein, the present invention resides in the novel features of form, construction, mode of operation and combination of parts presently described and understood by the claims.

### BRIEF DESCRIPTION OF THE DRAWINGS

In the drawings which illustrate the best modes presently contemplated for carrying out the present invention:

FIG. 1 is a top plan view of one embodiment of the present invention;

FIG. 2 is a side view the embodiment of the present invention shown in FIG. 1;

FIG. 3 is a perspective view of another embodiment of the present invention;

FIG. 4 is a perspective view of the embodiment shown in FIG. 3 utilizing pieces of tape as an alternative securing means;

FIG. 5 is a side view of an alternative embodiment of the present invention showing the use of a base portion; and

FIG. 6 is a side view of an alternative embodiment of the present invention showing the use of a belt to secure the deflector shield to the patient.

### DETAILED DESCRIPTION OF THE PREFERRED EMBODIMENTS

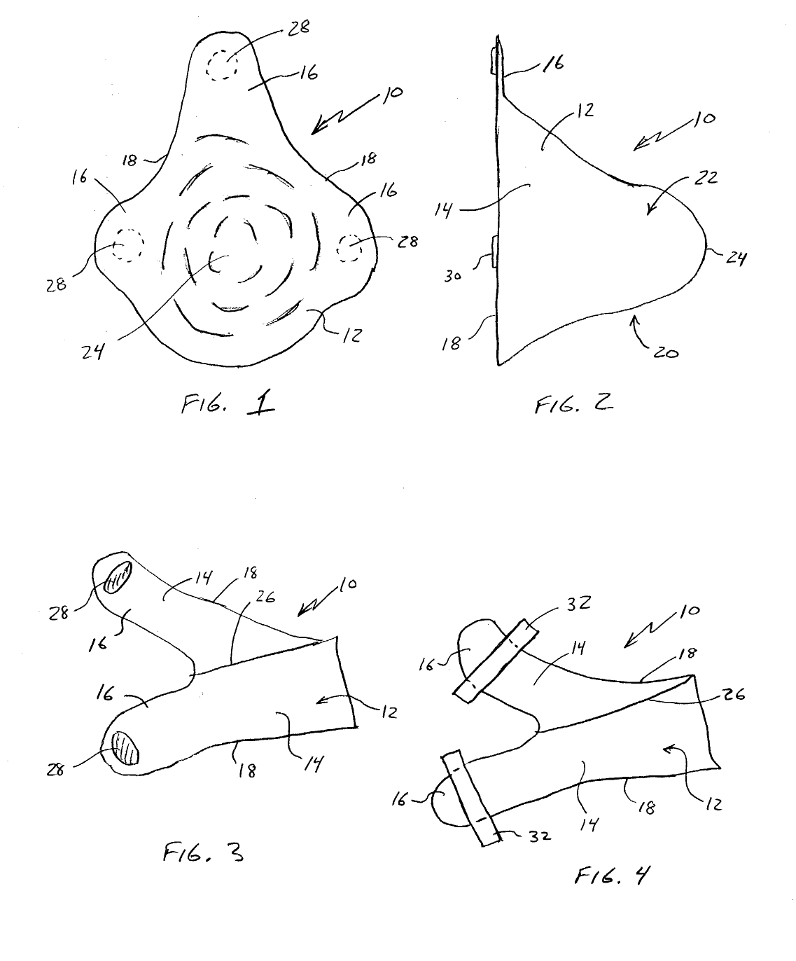

With reference to the figures where like elements have been given like numerical designations to facilitate the reader's understanding of the present invention, and particularly with reference to the embodiment of the present invention illustrated in FIGS. 1 through 4, the preferred embodiment of the present invention is set forth below. The enclosed figures and drawings are merely illustrative of the preferred embodiments and represent several different ways of configuring the present invention. Although specific components, materials, configurations and uses are illustrated, it should be understood that a number of variations to the components and to the configuration of those components described herein and in the accompanying figures can be made without changing the scope and function of the invention set forth herein.

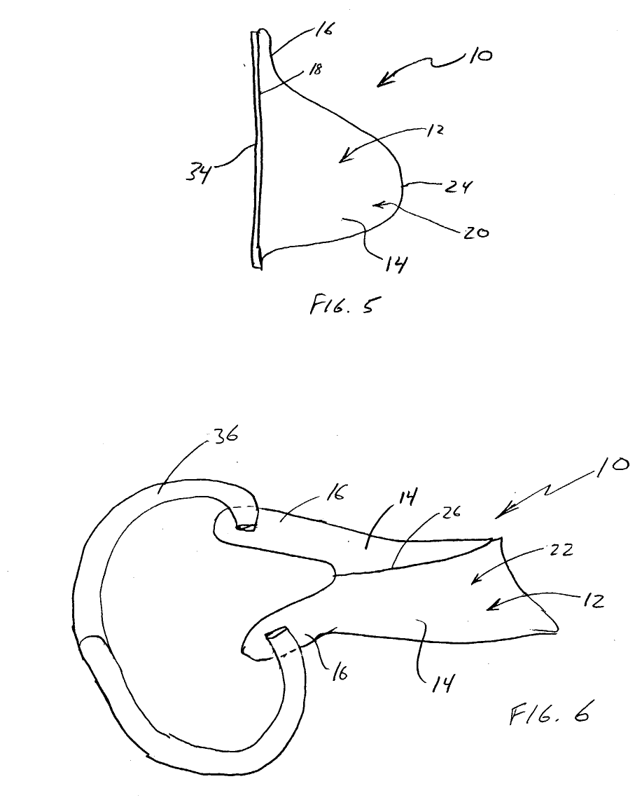

The urine deflector shield of the present invention is shown generally as 10 in FIGS. 1 through 6. The principal component of the present invention is the shaped cover 12 having one or more sides 14 and one or more wing members 16 extending from at least one of sides 14, as shown in FIGS. 1 through 4. Sides 14, having a bottom edge 18 thereon, form an outwardly projecting portion 20 of cover 12 into a tent-like configuration or enclosure 22 when sides 14 are spaced apart, as best shown in FIG. 3. As shown in FIGS. 1 and 2, cover 12 can be in the shape of a dome or dome-like structure having a generally circular cross-section that tapers to end 24. Alternatively, as shown in FIGS. 3 and 4, cover 12 can be in a generally pyramid shape having a top edge 26. Numerous other shapes are possible and suitable for cover 12, such as square, rectangle or triangular, as long as it is suitable for covering the male genitalia to accomplish the deflecting/shielding objectives set forth herein. The distance that end 24 or top edge 26 of outwardly projecting portion 20 projects outward from bottom edge 18 can vary significantly and still accomplish the objectives of the present invention. For instance, it is possible for projecting portion 20 to only be a few millimeters outward from bottom edge to one or more inches. The amount of this distance is determined by the desired separation between cover 12 and the male genitalia for which it is to cover. The present invention can be made in one size for both infant and adult males or be made in varying sizes as appropriate for infants or adults.

In the preferred embodiment, cover 12 is made out of a transparent plastic material so that it may accomplish the phototherapy objectives described above. A transparent material also serves the purpose of being able to see through cover 12 to easily determine if the male patient has discharged urine. The plastic material can be of various degrees of thickness and still accomplish the objectives set forth herein. For instance, cover 12 can be made out of a plastic material that is similar to that used for plastic baggies or other plastic bags. This material is generally relatively thin and, therefore, is more suitable for the pyramid-type shape of FIGS. 3 and 4 where spreading wing members 16 apart will cause top edge 26 to project outward from bottom edge 18 a sufficient distance. Alternatively, cover 12 may be made out of thicker plastic material that is sufficiently stiff enough for end 24 or top edge 26 to project outward (i.e., in an upright fashion on a male lying in a prone, face-up position) from bottom edge 18 on its own. In an alternative embodiment of the present invention 10, cover 12 can be made out of other materials that are suitable or which can be made suitable. For instance, cover 12 can be made out of a paper or cardboard that is either coated or covered with a fluid impenetrable material so that cover 12 will not become saturated with urine during use. Cover 12 can also be made out of a non-transparent material to provide a degree of modesty coverage for the patient.

The deflector shield 10 of the present invention can be made having a cover 12 that is foldable for ease of transport and storage. Deflector shield 10 can be folded up into a flat configuration that fits inside individual paper or plastic pouches for quick and ready access when needed. The deflector shield 10 can be prepared in a sterile manner and packaged in a way that maintains that sterile condition. These pouches, having deflector shields 10 therein, can be provided in an easy to access dispenser, such as a pre-cut or pre-shaped box. Alternatively, cover 12 can be rigid and provided in separate plastic bags or other containers that maintains the sterility of the prepared deflector shield 10.

In one embodiment of the present invention, urine deflector shield 10 as shown in FIGS. 1 and 2 can have a cover 12 that is made with a single circular cross-section side 14 that is approximately one and one-half to two and one-half inches diameter at bottom edge 18 and having end 24 projecting one to two inches from bottom edge 18. In the pyramid shape configuration of FIGS. 3 and 4, the base can be two to three inches across at bottom edge 18 and sides 14 can project top edge 26 such that it is one and one-half to two and one-half inches from bottom edge 18 when sides 14 are spaced apart. These dimensions are only representative of possible configurations for the deflector shield of the present invention. As stated above, end 24 or top edge 26 can extend various distances from bottom edge 18 and accomplish the objectives of the present invention.

Bottom edge 18 should be configured such that it does not have a sharp edge that could cut or otherwise cause irritation to the genital area, legs or stomach of the patient. If a thin plastic material is used, the sides 14 may be flexible enough that the bottom edge 18 in its unformed, cut condition is not going to cut or irritate the patient. If cover 12 is made out of a thicker material or a material that could have a sharp edge, then bottom edge 18 should be shaped and configured to avoid or minimize any such problems. For instance, bottom edge could be rounded to have a curved, non-sharp surface. Alternatively, bottom edge 18 could be configured to include an edge covering that is smooth enough to avoid cutting and irritation problems.

To prevent deflector shield 10 from being easily displaced by the patient or movement or treatment of the patient by healthcare workers, it should include some mechanism that can be used to secure it to the patient's body. In addition, the flow of urine from the patient could be sufficient to dislocate deflector shield 10 from its proper position, which could render it useless for its intended purpose (i.e., to deflect urine downward and prevent the spraying of urine). One such securing mechanism is the use of an adhesive material 28 located on the wing members 16, as shown on FIG. 1. Various adhesives, which are known in the art, are sufficient for removably securing deflector shield 10 to the human body. The preferred adhesive 28 is one that securely holds deflector shield 10 in place yet allows the deflector shield to be easily and painlessly (as much as possible) removed from the patient's body when it is not needed or it needs to be replaced. Particularly with regard to use of deflector shield 10 with infants, the adhesive 28 cannot tear or otherwise irritate the patient's sensitive skin when deflector shield 10 is removed. To prevent deflector shield 10 from sticking to its packaging or other items, adhesive 28 should be covered with an adhesive tab 30 that can be pulled off to expose the adhesive 28 when it is desired to secure deflector shield 10 on a patient. Each area having adhesive 28 can have its own adhesive tab 30 or a single tab layer can be used to expose all adhesive areas at once.

As an alternative to the use of adhesive 28 to secure deflector shield 10, tape 32 (shown in FIG. 4) can be used to secure wing members 16 to the patient's body. As shown in FIG. 4, tape 32 can extend across the top of wing members 16 with the adhesive side of tape 32 downward against wing member 16 and the patient's body. Another alternative is to use a two-sided tape (not shown) on the underside of deflector shield 10 at various positions, such as on wing members 16. One side of the tape can attach to wing member 16 and the other side of the two-sided tape can attach to the patient's body. As discussed above, the tape can have a covering on it so that it does not stick to items other than the patient's body. As with the adhesive 28, any tape used (i.e., whether tape 32 or two-sided tape) with deflector shield 10 should be of the type that is easily and painlessly as possible removed from the patient's body without causing any tears or irritation to the patient.

In an alternative embodiment of the present invention, shown in FIG. 5, deflector shield 10 includes a base portion 34 along bottom edge 18. Base portion 34 should be wider than the thickness of the material used for cover 12. In the preferred embodiment, base portion 34 is substantially wider (i.e., such as 4 mm wide) than the thickness of cover material. In this manner, base portion 34 provides a base for placing deflector shield 10 on the patient's body. Base portion 34 can be flexible so that it can follow the curves of the patient's body to ensure that no urine escapes past deflector shield 10 and to facilitate folding of deflector shield 10 into a convenient packaging arrangement (as described above). Base portion 34 can be rigid to further facilitate the outwardly projecting portion 20 extending outward from bottom edge 18. Base portion 34 can match or be in contrast to the flexible or rigid configuration of cover 12 (i.e., flexible cover with rigid base or rigid cover with flexible base or both flexible or rigid). Base portion 34 should generally follow the configuration of bottom edge 18 and wing members 16. If base portion 34 is used, adhesive 28, tape 32 or the double-sided tape can be located along all or part of base portion 34 so that base portion 34 can be secured to the patient's body. As discussed above, if adhesive is used, one or more adhesive tabs 30 should be used so the adhesive is only exposed when it is desired to secure deflector shield to a patient.

Another alternative embodiment of the deflector shield 10 of the present invention is shown in FIG. 6. In this embodiment, no adhesives or tape is used. Instead, the securing mechanism is a belt member 36 that connects to the wing members 16 of deflector shield 10 and goes around (i.e., under) the patient at or near the patient's waist area to securely hold deflector shield 10 in place above the genitals of the patient. The use of belt member 16 avoids potential problems associated with the use of adhesive 28 or tape 32, particularly on newborn infants. To facilitate being able to securely hold deflector shield 10 to various size patients, belt member 36 can include an adjustable mechanism (not shown), as commonly known in the art.

In use, the deflector shield 10 of the present invention is placed against the patient's body above the genital area so that any urine discharge will impact against in the inside of cover 12. If the embodiment shown in FIG. 1 is used, the adhesive tab 30 can be removed so the adhesive 28 on wing members 16 can be placed against the perineum area and the legs of the patient to fully enclose the genitals with tent-like configuration 22 of outwardly projecting portion 20. Alternatively, one of the wing members 16 can be positioned such that the adhesive is placed against the patient's body near the stomach. A less intrusive, as well as likely less annoying, positioning of deflector shield 10 can be obtained with the configuration of deflector shield 10 shown in FIG. 3. In this configuration, deflector shield 10 is positioned over the genital area by placing wing members 16 such that the adhesive 28 or tape 32 is located under the umbilical area of the patient's body. This type of configuration is likely to be preferred for use with infants. If base portion 34 is used, the adhesive tab 30 or cover is removed from the base portion 34 to expose the adhesive 28 or double-sided tape so that the base portion 34 can be positioned properly above the genital area of the patient. If the belt member 36 configuration of FIG. 6 is used, the belt member 36 is placed around (i.e., under) the patient's body at or about the waist or pelvic area and attached to one or more wing members 16 (depending if belt member 36 comes pre-attached to one or more wing members 16). The belt member 36 is adjusted to securely place deflector shield 10 over the patient's genitals. Once in place, any urine discharge will impact against the inside of cover 12 and be directed downward where it can be collected for analysis (i.e., measured) or disposal. In this manner, the spraying of urine onto the patient or to healthcare workers and hospital equipment will be eliminated or, at least, minimized. This will reduce or possibly eliminate the problems associated with urine spraying, including cross-contamination of diseases amongst patients, disease exposure to the healthcare workers and the unpleasantness of being sprayed with urine.

While there is shown and described herein certain specific alternative forms of the invention, it will be readily apparent to those skilled in the art that the invention is not so limited, but is susceptible to various modifications and rearrangements in design and materials without departing from the spirit and scope of the invention. In particular, it should be noted that the present invention is subject to modification with regard to the dimensional relationships set forth herein and modifications in assembly, materials, size, shape, and use.

What is claimed is:

1. A urine deflector shield, comprising:
a shaped cover having one or more sides forming an outwardly projecting portion into a tent-like configuration when said one or more sides are spaced apart, each of said one or more sides having a bottom edge thereon;
one or more wing members extending from at least one of said one or more sides; and
a securing means on at least one of said wing members for removably securing said cover to a human body to deflect a stream of urine therefrom.

2. The urine deflector shield according to claim 1, wherein said cover is made out of a transparent plastic material.

3. The urine deflector shield according to claim 1, wherein said cover is foldable.

4. The urine deflector shield according to claim 1, wherein said cover is rigid.

5. The urine deflector shield according to claim 1 further comprising a base portion at said bottom edge.

6. The urine deflector shield according to claim 5, wherein said base portion is rigid.

7. The urine deflector shield according to claim 1, wherein said bottom edge is shaped and configured to be smooth.

8. The urine deflector shield according to claim 1, wherein said securing means comprises an adhesive.

9. The urine deflector shield according to claim 8, wherein said adhesive is covered by a removable tab member.

10. The urine deflector shield according to claim 1, wherein said securing means comprises one or more pieces of tape.

11. The urine deflector shield according to claim 1, wherein said securing means comprises a belt member.

12. A urine deflector shield, comprising:
a shaped transparent plastic cover having one or more sides forming an outwardly projecting portion into a tent-like configuration when said one or more sides are spaced apart, each of said one or more sides having a bottom edge thereon, said cover being foldable;
one or more wing members extending from at least one of said one or more sides; and
a securing means on at least one of said wing members for removably securing said cover to a human body to deflect a stream of urine therefrom.

13. The urine deflector shield according to claim 12 further comprising a base portion at said bottom edge.

14. The urine deflector shield according to claim 12, wherein said securing means comprises an adhesive.

15. The urine deflector shield according to claim 14, wherein said adhesive is covered by a removable tab member.

16. The urine deflector shield according to claim 12, wherein said securing means comprises one or more pieces of tape.

17. The urine deflector shield according to claim 12, wherein said securing means comprises a belt member.
